(19) United States
(12) Patent Application Publication
(10) Pub. No.: US 2023/0416720 A1
(43) Pub. Date: Dec. 28, 2023

(54) CHITOSAN-MODIFIED OPTO-HYDRODYNAMIC MICROMOTOR AND PREPARATION METHOD AND USE THEREOF

(71) Applicant: Jinan University, Guangzhou City (CN)

(72) Inventors: Hongbao Xin, Guangzhou City (CN); Baojun Li, Guangzhou City (CN); Jianyun Xiong, Guangzhou City (CN); Yang Shi, Guangzhou City (CN); Ting Pan, Guangzhou City (CN)

(73) Assignee: Jinan University, Guangzhou City, GD (CN)

(21) Appl. No.: 17/933,679

(22) Filed: Sep. 20, 2022

(30) Foreign Application Priority Data
Jun. 22, 2022 (CN) 202210710440.0

(51) Int. Cl. C12N 11/10; C12N 5/09; A01N 65/03

(52) U.S. Cl. CPC C12N 11/10; C12N 5/0693; A01N 65/03

(57) ABSTRACT

The present disclosure provides a chitosan-modified opto-hydrodynamic micromotor and a preparation method and use thereof, and belongs to the technical field of micromotors. The chitosan-modified opto-hydrodynamic micromotor provided by the present disclosure is composed of Phaeodactylum tricornutum Bohlin and a chitosan solution. The chitosan-modified opto-hydrodynamic micromotor provided by the present disclosure has high biocompatibility, can non-invasively remove biological threats in a microenvironment containing cells with a sterilization rate reaching about 98%, and can achieve high-efficiency sterilization without affecting cell viability.

### CROSS REFERENCE TO RELATED APPLICATION

This patent application claims the benefit and priority of Chinese Patent Application No. 202210710440.0, filed on Jun. 22, 2022, the disclosure of which is incorporated by reference herein in its entirety as part of the present application.

### TECHNICAL FIELD

The present disclosure belongs to the technical field of micromotors, and in particular, to a chitosan-modified opto-hydrodynamic micromotor and a preparation method and use thereof.

### BACKGROUND ART

Biological threats, such as viruses, mycoplasmas, and pathogenic bacteria, are notorious foreign contaminants in cell culture and biological microenvironments. These organisms can alter the physiology of cultured cells and the structure of recombinant biomolecules. In addition, because of the rapid reproduction ability of bacteria, even a small number of bacteria in the biological microenvironments can pose a huge threat or even disaster to different biomedical applications from basic cell culture to biomanufacturing and recombinant biotherapy. Therefore, non-invasive removal and killing of biological threats is essential.

Traditional sterilization methods include ultraviolet sterilization, 75% alcohol sterilization, and antibiotic sterilization. However, high doses of fungicides are required by using the above sterilization methods to achieve effective sterilization, which is lack of selectivity, and is likely to cause irreversible damage to cells and other biological samples. Although the development of bactericidal nanomaterials has added a new dimension to the removal and sterilization effects of biological threats, this sterilization method is static and passive. Therefore, more and more researchers are interested in micronano motors that can convert external energy into their own movement. For example, chemically driven micromotors can remove biological contaminants by generating chemically induced air bubbles to propel themselves in movement. However, chemical reactions within the microenvironment hinder its further biomedical applications. In addition, magnetron micromotors are also widely used to remove biological threats. However, additional magnetic materials are often required by these magnetron micromotors to respond to the magnetic source. Therefore, more and more researchers are interested in the direct use of micromotors with self-propelled microorganisms. However, these motile microorganisms can affect cell growth and metabolism during culture. Conversely, the cell culture medium affects and disrupts the movement characteristics of the microorganisms, making it impossible to further play the role of micromotors in cell culture. Therefore, it is necessary to design an intelligent, active, and biocompatible micromotor platform that can be directly used in the biological microenvironment of cell culture to remove biological threats.

### SUMMARY

In view of this, an objective of the present disclosure is to provide a chitosan-modified opto-hydrodynamic micromotor, which has high biocompatibility and can non-invasively remove and kill biological threats in the cellular environment.

To achieve the above objective, the present disclosure provides the following technical solutions.

The present disclosure provides a chitosan-modified opto-hydrodynamic micromotor, including the following raw materials: Phaeodactylum tricornutum Bohlin and a chitosan solution.

Preferably, the chitosan solution may have a concentration of 0.2-0.5 mg/mL.

Preferably, the Phaeodactylum tricornutum Bohlin may have a size of (6.3-10.9)) μm×(1.1-2.7)) μm.

The present disclosure further provides a preparation method of the above opto-hydrodynamic micromotor, including the following steps: mixing the chitosan solution with the Phaeodactylum tricornutum Bohlin for combination for at least 4 h at 100-250 rpm, and removing a supernatant to obtain the chitosan-modified opto-hydrodynamic micromotor.

Preferably, the chitosan solution may be obtained by mixing a chitosan solid with glacial acetic acid with a concentration of 2%.

The present disclosure further provides a use of the above opto-hydrodynamic micromotor or the above preparation method in cell culture.

The present disclosure further provides a use of the above opto-hydrodynamic micromotor or the above preparation method in removal of biological threats in cell culture.

The present disclosure further provides a use of the above opto-hydrodynamic micromotor or the above preparation method in improvement of cell viability or improvement of a cell survival rate.

The present disclosure further provides a method for non-invasively removing biological threats in cell culture, including the following steps: mixing the opto-hydrodynamic micromotor prepared by the above preparation method or the above opto-hydrodynamic micromotor with a cell culture medium, and driving the opto-hydrodynamic micromotor to rotate by an annular optical trap.

Preferably, the annular optical trap may have a power of 20-100 mW and a frequency of 6,000-9,000 Hz.

The present disclosure has the following beneficial effects:

The chitosan-modified opto-hydrodynamic micromotor provided by the present disclosure has high biocompatibility, can non-invasively remove biological threats in a microenvironment containing cells with a sterilization rate reaching about 98%, and can achieve high-efficiency sterilization without affecting cell viability.

### BRIEF DESCRIPTION OF THE DRAWINGS

FIG. 1 is a preparation flow chart of a chitosan-modified opto-hydrodynamic micromotor of the present disclosure;

FIG. 2 shows biocompatibility verification results of the chitosan-modified opto-hydrodynamic micromotor, in which a diagram a shows fluorescence images of co-culture of human promyelocytic leukemia cells and the chitosan-modified opto-hydrodynamic micromotor, and a diagram b shows cell viability test results after co-culture for different times;

FIG. 3A-B shows results of non-invasive removal of biological threats by the chitosan-modified opto-hydrodynamic micromotor, in which FIG. 3A is a flow pattern diagram of non-invasive removal, and FIG. 3B shows removal rate effects of non-invasive removal of different types of biological threats, and a first row, a second row, and a third row from top to bottom represent removal effects of a virus model (150 nm polystyrene particles), Escherichia coli, and mycoplasma respectively;

FIG. 4A-C shows verification results of sterilization performance of the chitosan-modified opto-hydrodynamic micromotor, in which FIG. 4A shows fluorescence images of mixing of different groups for 10 min, FIG. 4B shows sterilization efficiency of mixing of different groups for 20 min, and FIG. 4C shows fluorescence intensities of different groups;

FIG. 5 shows sterilization efficiency of the opto-hydrodynamic micromotor modified by chitosan with different concentrations; and

FIG. 6A-C shows effects of different groups on cell viability in an HL-60 cell culture medium contaminated with Escherichia coli, in which FIG. 6A is a flow chart of reactions of different groups, FIG. 6B shows fluorescence images of cell viability treated with an opto-hydrodynamic micromotor without chitosan modification, and FIG. 6C shows fluorescence images of cell viability treated with the chitosan-modified opto-hydrodynamic micromotor.

### DETAILED DESCRIPTION OF THE EMBODIMENTS

The present disclosure provides a chitosan-modified opto-hydrodynamic micromotor, including the following raw materials: Phaeodactylum tricornutum Bohlin and a chitosan solution.

The present disclosure has no special limitations on specific sources of the Phaeodactylum tricornutum Bohlin and chitosan. In the present disclosure, the chitosan solution has a concentration of preferably 0.2-0.5 mg/mL, more preferably 0.3-0.4 mg/mL, and the Phaeodactylum tricornutum Bohlin has a size of preferably (6.3-10.9) μm×(1.1-2.7)) μm, more preferably 8 μm×1.8) μm. The chitosan-modified opto-hydrodynamic micromotor provided by the present disclosure has high biocompatibility, and can achieve controlled movement under the action of an optical trap, and after moving to a designated position, non-invasive and effective collection of biological threats around cells can be achieved by generating a mild flow field using optical and hydrodynamic forces. After the effective collection is achieved, the sterilization efficiency is greatly improved due to the close contact between the biological threats and the chitosan. Therefore, the chitosan-modified opto-hydrodynamic micromotor provided in the present disclosure can not only efficiently collect and kill bacteria, but also does not affect the biological activity and cell viability of the cells themselves.

The present disclosure further provides a preparation method of the above opto-hydrodynamic micromotor, including the following steps: the chitosan solution is mixed with the Phaeodactylum tricornutum Bohlin for combination for at least 4 h at 100-250 rpm, and supernatant is removed to obtain the chitosan-modified opto-hydrodynamic micromotor.

In the present disclosure, the chitosan solution is preferably obtained by mixing a chitosan solid with glacial acetic acid with a concentration of 2%. After the chitosan solution is mixed with the Phaeodactylum tricornutum Bohlin, it is preferable to conduct shaking culture on a shaking table. The rotational speed of the shaking table is preferably 150-230 rpm, more preferably 180-200 rpm, and the shaking culture is conducted on the shaking table at preferably 37° C. The present disclosure has no special limitations on a mixing ratio of the chitosan solution and the Phaeodactylum tricornutum Bohlin. The present disclosure has no special limitations on specific steps of removing the supernatant. In a specific example of the present disclosure, the supernatant is removed by centrifugation, and the centrifugation is preferably conducted at 1,500 rpm for 10 min.

The present disclosure further provides a use of the above opto-hydrodynamic micromotor or the above preparation method in cell culture, in removal of biological threats in cell culture, and in improvement of cell viability or improvement of a cell survival rate.

The present disclosure has no special limitations on the specific type of cell culture, including suspension cell culture and adherent cell culture. The present disclosure has no special limitations on the specific type of cells, and the opto-hydrodynamic micromotor of the present disclosure is suitable for removal of biological threats in various types of cell culture without affecting the biological activity and viability of the cells themselves.

The present disclosure further provides a method for non-invasively removing biological threats in cell culture, including the following steps: the opto-hydrodynamic micromotor prepared by the above preparation method or the above opto-hydrodynamic micromotor is mixed with a cell culture medium, and the opto-hydrodynamic micromotor is driven to rotate by an annular optical trap.

In the present disclosure, the annular optical trap that drives the rotation of the opto-hydrodynamic micromotor has a power of preferably 20-100 mW, more preferably mW, most preferably 50 mW. The annular optical trap has a frequency of 6,000-9,000 Hz, more preferably 7,000-8,500 Hz, most preferably 8,000 Hz. In the present disclosure, it is preferable to apply an annular optical trap to the chitosan-modified opto-hydrodynamic micromotor by a standard optical tweezers system based on 1,064 nm. Due to the high-speed rotation of the chitosan-modified Phaeodactylum tricornutum Bohlin, highly localized flow fields and hydrodynamic vortices are induced around the arms of the chitosan-modified Phaeodactylum tricornutum Bohlin. The optical trapping force is combined with the hydrodynamic force (opto-hydrodynamics) to constitute a controllable and movable chitosan-modified opto-hydrodynamic micromotor with a rotational speed of 200 rpm. By moving the chitosan-modified opto-hydrodynamic micromotor along the predefined trajectory of the optical trap, the chitosan-modified opto-hydrodynamic micromotor can be controllably navigated to a specified position. Targeted removal of biological threats can be conducted in the presence of cells without affecting the viability of cultured cells. In addition, since the chitosan has bactericidal ability, after the opto-hydrodynamic micromotor achieves effective collection of biological threats, due to the close contact between the chitosan on the surface of the opto-hydrodynamic micromotor and the biological threats, the biological threats can be efficiently killed, and the survival rate of the cells is greatly improved, which brings great promise for cell-based biomanufacturing and single-cell therapy.

The technical solution provided by the present disclosure will be described in detail below with reference to examples, but they should not be construed as limiting the protection scope of the present disclosure.

### Example 1

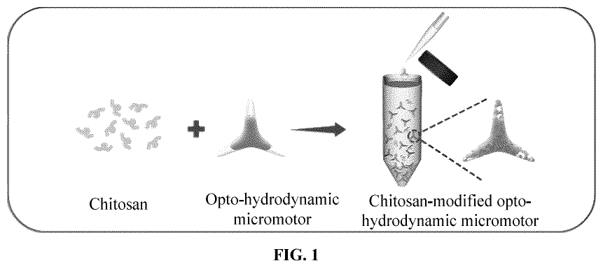

Chitosan was dissolved in 2% glacial acetic acid to prepare a chitosan solution with a concentration of 0.2 mg/mL. The 0.2 mg/mL chitosan solution was mixed with Phaeodactylum tricornutum Bohlin (with a size of approximately 8 μm×1.8) μm and an angle of 120°). A mixture was placed on a constant-temperature shaking table at 200 rpm and shaken at 37° C. for 4 h. Finally, the mixture was centrifuged at 1,500 rpm for 10 min, and supernatant was removed to obtain a chitosan-modified opto-hydrodynamic micromotor, as shown in FIG. 1.

### Example 2

Chitosan was dissolved in 2% glacial acetic acid to prepare a chitosan solution with a concentration of 0.5 mg/mL. The 0.5 mg/mL chitosan solution was mixed with Phaeodactylum tricornutum Bohlin. A mixture was placed on a constant-temperature shaking table at 100 rpm and shaken at 37° C. for 5 h. Finally, the mixture was centrifuged at 1,500 rpm for 10 min, and supernatant was removed to obtain a chitosan-modified opto-hydrodynamic micromotor.

### Example 3

Chitosan was dissolved in 2% glacial acetic acid to prepare a chitosan solution with a concentration of 0.3 mg/mL. The 0.3 mg/mL chitosan solution was mixed with Phaeodactylum tricornutum Bohlin. A mixture was placed on a constant-temperature shaking table at 250 rpm and shaken at 37° C. for 4 h. Finally, the mixture was centrifuged at 1,500 rpm for 10 min, and supernatant was removed to obtain a chitosan-modified opto-hydrodynamic micromotor.

### Example 4

Biocompatibility Verification of Chitosan-Modified Opto-Hydrodynamic Micromotor

Human promyelocytic leukemia cells (HL-60 cells) and HeLa cells were co-cultured with the chitosan-modified opto-hydrodynamic micromotor in Example 1 in a Gibco Dulbecco's Modified Eagle Medium (DMEM) supplemented with 4,500 mg/L of various amino acids and glucose, 10% fetal bovine serum (FBS), and 1% penicillin/streptomycin (P/S), and mixed overnight in a 37° C. incubator containing 5% CO₂. The cell viability was tested at 6 h, 12 h, 18 h, and 24 h of co-culture. The specific test method for cell viability was as follows: dual fluorescent calcein-acetoxymethyl (AM)/propidium iodide (PI) (purchased from Jiangsu KeyGEN BioTECH Co., Ltd.) was used to test the viability of the HL-60 cells. In the experiment, 2 μL of AM and PI dyes were added to the co-cultured cells for 25 min. For living cells, AM can react with esterases, which fluoresces the living cells strongly green. In contrast, PI cannot pass through living cell membranes, but can reach the nucleus through disordered regions of dead cell membranes. Thus, the DNA double helix structure within the cell binds to PI, causing the dead cell to fluoresce red. Finally, the viability of the HL-60 cells was determined by the red and green fluorescence of the HL-60 cells, and the biocompatibility of the chitosan-modified opto-hydrodynamic micromotor was verified.

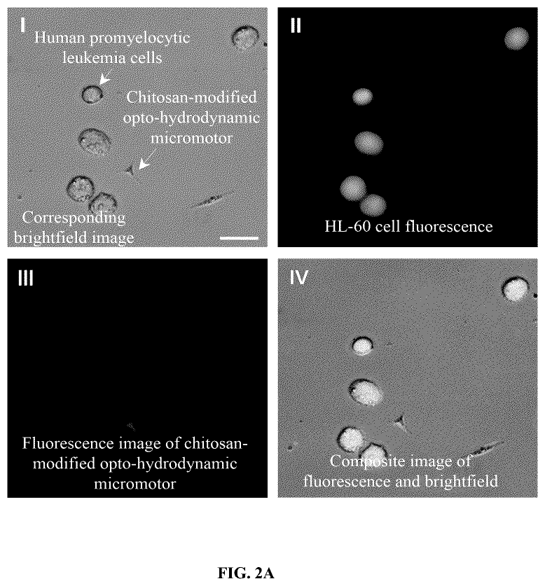

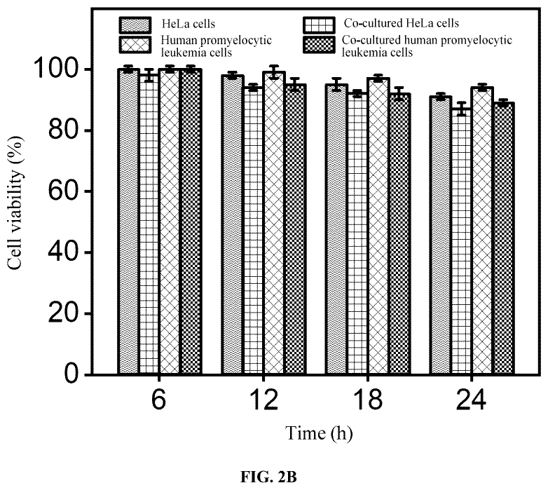

The results are shown in FIG. 2. It shows that the chitosan-modified opto-hydrodynamic micromotor of the present disclosure has good biocompatibility when mixed with various cells such as adherent cells and suspension cells.

### Example 5

Preparation of biological threats: virus model-polystyrene particles (particle size of 150 nm) were purchased from Shanghai Macklin Biochemical Co., Ltd. The virus model was first suspended in deionized water and sonicated for 10-15 min to obtain a monodisperse suspension with a final concentration of about 4×10³ to 8×10³ particles per microliter. After preparation, the cell culture suspension (containing HL-60 cells) contaminated by the 150 nm polystyrene suspension was mixed with the chitosan-modified opto-hydrodynamic micromotor in Example 2, and injected into a microfluidic chamber through a disposable syringe for subsequent experiments.

Pathogenic bacteria (Escherichia coli and Staphylococcus aureus) were grown in lysogenic broth for 3-4 h in a shaking table at 180 rpm at 37° C. The bacteria were washed with phosphate buffer and diluted with phosphate buffer to obtain a suitable concentration of about 2×10⁴ to 5×10⁴ bacteria per microliter. After preparation, the cell culture suspension (containing HL-60 cells) contaminated by the pathogenic bacteria suspension was mixed with the chitosan-modified opto-hydrodynamic micromotor in Example 2, and injected into the microfluidic chamber through a disposable syringe for subsequent experiments.

The cell culture suspension contaminated by mycoplasma (1 mL, containing HL-60 cells) was mixed with suspension of the chitosan-modified opto-hydrodynamic micromotor in Example 2. A mixture of the cell suspension contaminated by the mycoplasma and the chitosan-modified opto-hydrodynamic micromotor in Example 2 was injected into the microfluidic chamber through a disposable syringe for subsequent experiments.

Preparation of microfluidic chamber: a silicone elastomer and a curing agent were mixed at a weight ratio of 10:1, and a solution was placed in a vacuum pump to remove air bubbles generated in the mixing process. 50 μm-thick SU8-3005 photoresist was coated on a clean Si/SiO₂ wafer and exposed to a standard lithography device to prepare a master mold for the microfluidic chamber. Bubble-free polydimethylsiloxane (PDMS) was cast on the SU8 photoresist master mold and cured at 70° C. for 3 h. After the curing process, the PDMS was peeled from the mold and bonded to the glass slide by O₂ plasma bonding.

Non-invasive removal of biological threats using chitosan-modified opto-hydrodynamic micromotor:

When the above chitosan-modified opto-hydrodynamic micromotor and the biological threats were mixed, the suspension of the two had a volume ratio of 4:1, and a mixed suspension was obtained. The mixed suspension was injected into the microfluidic chamber, that is, the mixed suspension was injected into the microfluidic channel using a syringe. The microfluidic channel was put on the three-dimensional operating platform of the standard optical tweezers system. An annular scanning optical trap with an optical power of 50 mW and an optical frequency of 8,000 Hz was applied to the chitosan-modified opto-hydrodynamic micromotor to drive the chitosan-modified opto-hydrodynamic micromotor to rotate. Due to the rotation of the chitosan-modified opto-hydrodynamic micromotor (200 rpm), local flow fields and hydrodynamic vortices were generated around the chitosan-modified opto-hydrodynamic micromotor, and randomly distributed biological threats were collected around the arms of the chitosan-modified opto-hydrodynamic micromotor. The collected biological threats could be navigated and swept to the designated position by the chitosan-modified opto-hydrodynamic micromotor. Then by turning off the trapping laser, the local flow fields around the arms of the chitosan-modified opto-hydrodynamic micromotor disappeared, allowing the collected biological threats to be released at the designated location. Subsequently, switching the annular scanning optical trap to the central optical trap with a relatively low power (5 mW) could navigate the chitosan-modified opto-hydrodynamic micromotor to other locations for reuse in subsequent experiments.

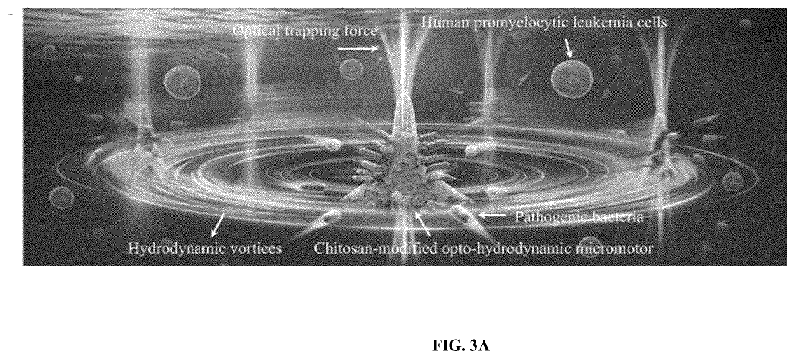

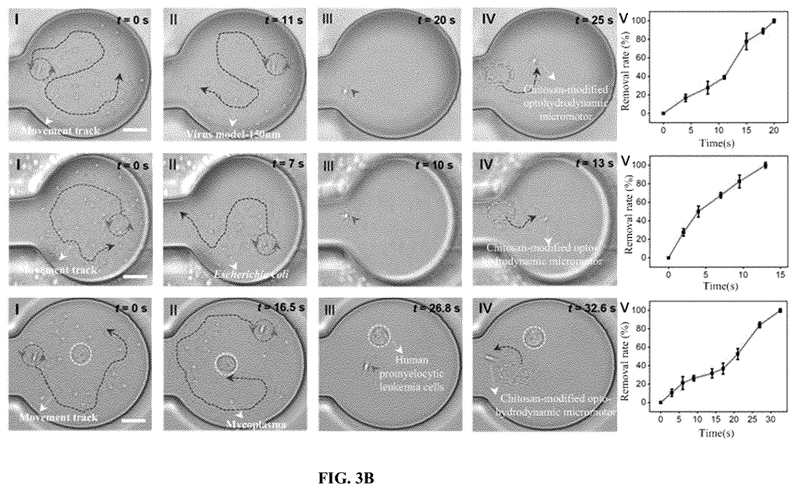

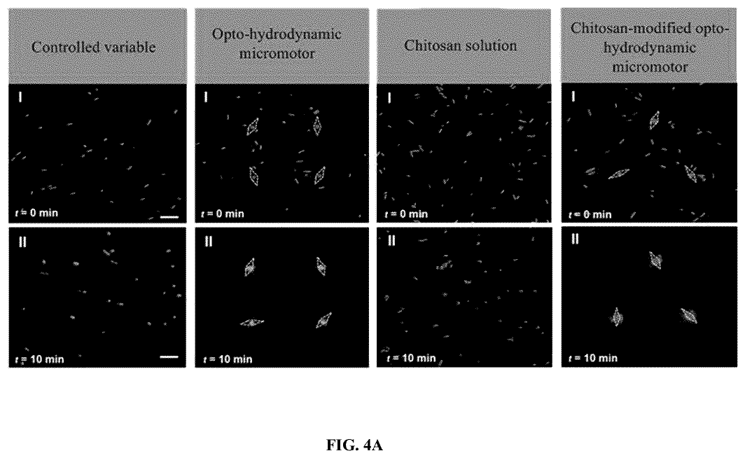

The experimental results are shown in FIG. 3A-B. As can be seen from FIG. 3A-B, the chitosan-modified opto-hydrodynamic micromotor of the present disclosure can achieve non-invasive removal of various types of biological threats in the cellular environment, such as virus model-150 nm polystyrene particles, pathogenic bacteria, and mycoplasma.

### Example 6

Verification of Sterilization Performance of Chitosan-Modified Opto-Hydrodynamic Micromotor

Biological threats always greatly threatened cell culture and hindered further single-cell analysis and therapy. Although the chitosan-modified opto-hydrodynamic micromotor had non-invasive and efficient collection and removal of the biological threats in biological microenvironments, further non-invasive and efficient killing of contaminated biological threats in the biological microenvironments was important to ensure further single-cell analysis and therapy. In order to verify that the chitosan-modified opto-hydrodynamic micromotor of the present disclosure had bactericidal ability, in the present example, the chitosan-modified opto-hydrodynamic micromotor in Example 1 was mixed with Escherichia coli with excellent activity to test the bactericidal ability of the chitosan-modified opto-hydrodynamic micromotor.

The experiment was divided into four groups, namely a control group (a blank control group, containing only Escherichia coli), an opto-hydrodynamic micromotor group (containing Escherichia coli and a bare opto-hydrodynamic micromotor, the bare opto-hydrodynamic micromotor was only the Phaeodactylum tricornutum Bohlin in Example 1 with modification of the chitosan in Example 1), a chitosan solution group (containing Escherichia coli and the chitosan solution in Example 1), and a chitosan-modified opto-hydrodynamic micromotor group (containing Escherichia coli and the chitosan-modified opto-hydrodynamic micromotor in Example 1). The preparation of the microfluidic chamber and the specific steps for the annular scanning optical trap were the same as those in Example 5.

The bactericidal properties of the chitosan-modified opto-hydrodynamic micromotor were demonstrated by testing the activity of Escherichia coli by a bacterial viability kit (DMAO: 9-Octadecen-1-amine,N,N-dimethyl-(9Z)—; and EthD-3: Ethidium Homodimer 3 dyes were purchased from Yeasen Biotechnology (Shanghai) Co., Ltd., China). In the experiment, 2 μL of DMAO and EthD-3 dye were added to 100 μL of the mixture and mixed together for 20 min to characterize the final viability of the Escherichia coli. The DMAO dye (green) was used to label live bacteria, while the EthD-3 dye (red) could only penetrate damaged bacteria and was used to label dead Escherichia coli.

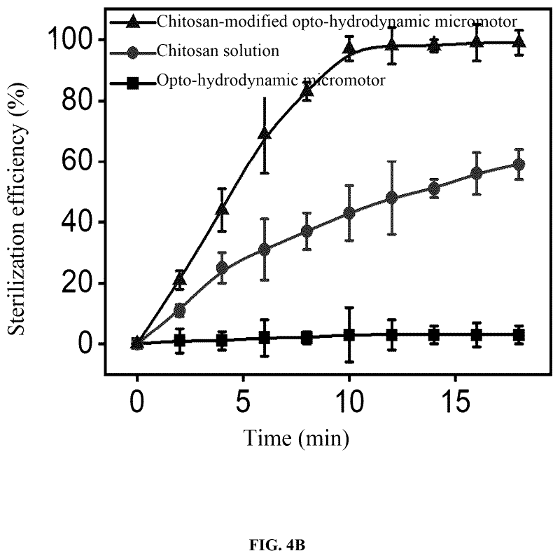

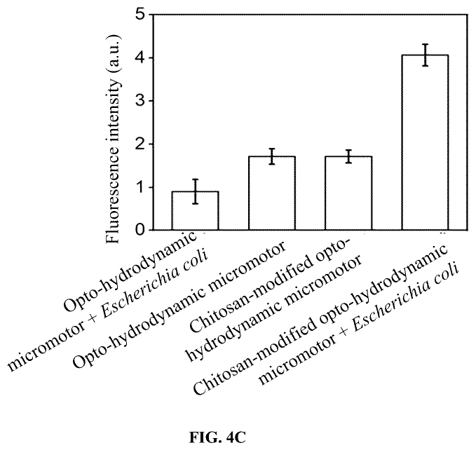

The results are shown in FIG. 4A-C. As can be seen from FIG. 4A-C, 10 min after the Escherichia coli is collected, the bare opto-hydrodynamic micromotor emits yellow fluorescence, which is a combination of red (Phaeodactylum tricornutum Bohlin) and green (live Escherichia coli) fluorescence. This phenomenon suggests that the opto-hydrodynamic micromotor cannot cause the death of Escherichia coli. In the group treated only with the chitosan solution, only about 40% of the fluorescence of Escherichia coli is red, while the others are still green after 10 min of treatment, indicating that only about 40% of the bacteria are killed by the chitosan solution. For the chitosan-modified opto-hydrodynamic micromotor, all fluorescence is red, indicating that 98% of the bacteria are killed after 10 min. It can be seen that the chitosan-modified opto-hydrodynamic micromotor has efficient sterilization performance.

### Example 7

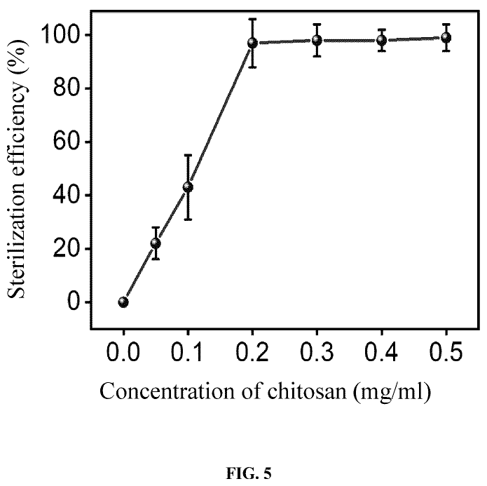

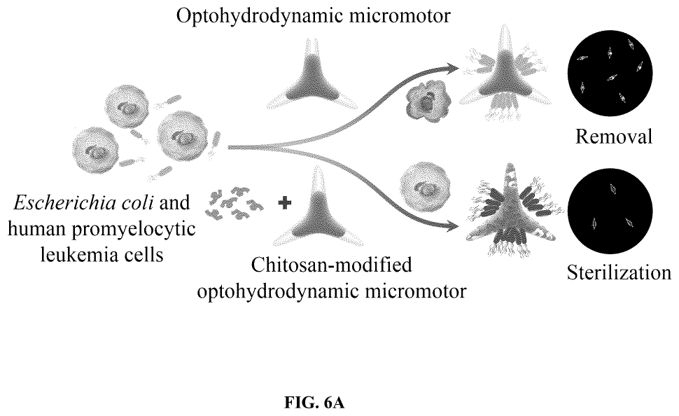

Chitosan was dissolved in 2% glacial acetic acid to prepare chitosan solutions with concentrations of 0 mg/mL, 0.1 mg/mL, 0.2 mg/mL, 0.3 mg/mL, 0.4 mg/mL, and mg/mL. The other steps were the same as those in Example 1, and opto-hydrodynamic micromotors modified with different concentrations of chitosan solutions were prepared. The opto-hydrodynamic micromotors modified with different concentrations of chitosan solutions were mixed with Escherichia coli with excellent activity to test the bactericidal ability of the opto-hydrodynamic micromotors modified with different concentrations of chitosan. The specific steps for bactericidal ability test were the same as those in Example 6. The results are shown in FIG. 5. When the concentration of the chitosan reaches 0.2 mg/ml, the sterilization efficiency of the chitosan-modified opto-hydrodynamic micromotor reaches 98%.

### Example 8

Guarantee of Chitosan-Modified Opto-Hydrodynamic Micromotor for Cell Viability after Efficient Sterilization

The chitosan-modified opto-hydrodynamic micromotor in Example 1 and the opto-hydrodynamic micromotor without chitosan modification (the difference from Example 1 was that the chitosan was not used, and the rest were the same as those in Example 1) were mixed with the HL-60 cell culture medium contaminated with Escherichia coli. The preparation of the microfluidic chamber and the specific steps for the annular scanning optical trap were the same as those in Example 5. The test method for bacterial viability was the same as that in Example 6, and the test method for viability of HL-60 cells was the same as that in Example 4.

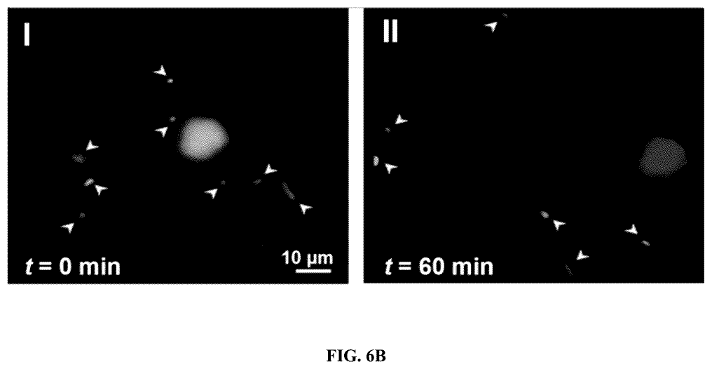

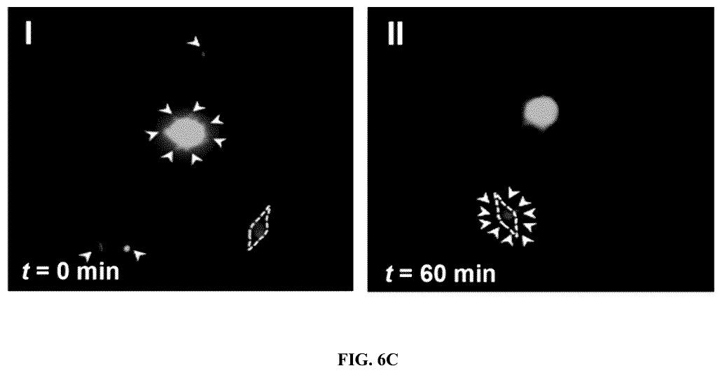

The results are shown in FIG. 6A-C. In the opto-hydrodynamic micromotor group without chitosan modification, the live HL-60 cells (green) were infected with live bacteria, and the cells died (red) after 60 min. However, by using the chitosan-modified opto-hydrodynamic micromotor for bacterial removal and sterilization, even if the microenvironment was contaminated with the Escherichia coli, the HL-60 cells were not infected with bacteria, and the cell viability was not affected after 60 min (green fluorescence).

The above descriptions are merely preferred implementations of the present disclosure. It should be noted that those of ordinary skill in the art may further make several improvements and modifications without departing from the principle of the present disclosure, but such improvements and modifications should be deemed as falling within the protection scope of the present disclosure.

1. A chitosan-modified opto-hydrodynamic micromotor, comprising the following raw materials: Phaeodactylum tricornutum Bohlin and a chitosan solution.

2. The opto-hydrodynamic micromotor according to claim 1, wherein the chitosan solution has a concentration of 0.2-0.5 mg/mL.

3. The opto-hydrodynamic micromotor according to claim 1, wherein the Phaeodactylum tricornutum Bohlin has a size of (6.3-10.9) μm×(1.1-2.7) μm.

4. A preparation method of the opto-hydrodynamic micromotor according to claim 1, comprising the following steps: mixing the chitosan solution with the Phaeodactylum tricornutum Bohlin for combination for at least 4 h at 100-250 rpm, and removing a supernatant to obtain the chitosan-modified opto-hydrodynamic micromotor.

5. The preparation method according to claim 4, wherein the chitosan solution is obtained by mixing a chitosan solid with glacial acetic acid with a concentration of 2%.

6. The preparation method according to claim 11, wherein the chitosan solution is obtained by mixing a chitosan solid with glacial acetic acid with a concentration of 2%.

7. (canceled)

8. (canceled)

9. A method for non-invasively removing biological threats in cell culture, comprising the following steps: mixing the optohydrodynamic micromotor according to claim 1 with a cell culture medium, and driving the optohydrodynamic micromotor to rotate by an annular optical trap.

10. The method according to claim 9, wherein the chitosan solution has a concentration of 0.2-0.5 mg/mL.

11. The preparation method according to claim 4, wherein the chitosan solution has a concentration of 0.2-0.5 mg/mL.

12. The preparation method according to claim 4, wherein the Phaeodactylum tricornutum Bohlin has a size of (6.3-10.9) μm×(1.1-2.7) μm.

13. The preparation method according to claim 12, wherein the chitosan solution is obtained by mixing a chitosan solid with glacial acetic acid with a concentration of 2%.

14. The method according to claim 9, wherein the Phaeodactylum tricornutum Bohlin has a size of (6.3-10.9) μm×(1.1-2.7) μm.

15. The method according to claim 9, wherein the annular optical trap has a power of mW and a frequency of 6,000-9,000 Hz.

16. The method according to claim 10, wherein the annular optical trap has a power of 20-100 mW and a frequency of 6,000-9,000 Hz.

17. The method according to claim 14, wherein the annular optical trap has a power of 20-100 mW and a frequency of 6,000-9,000 Hz.
